(19) United States
(12) Patent Application Publication
(10) Pub. No.: US 2025/0049829 A1
(43) Pub. Date: Feb. 13, 2025

(54) NOVEL THERAPEUTIC MOLECULES TARGETING THE H-RAS ISOFORM WITH G12D MUTATION INVOLVED IN CANCER FORMATION

(71) Applicant: ISTANBUL MEDIPOL UNIVERSITESI, Istanbul (TR)

(72) Inventors: Ramazan KASMER, Istanbul (TR); Metehan ILTER, Istanbul (TR); Özge SENSOY, Istanbul (TR); Nihal KARAKAS, Istanbul (TR); Ozan TOPÇU, Istanbul (TR)

(21) Appl. No.: 18/719,989

(22) PCT Filed: Dec. 8, 2022
(86) PCT No.: PCT/TR2022/051452

§ 371 Date: Jun. 14, 2024

(30) Foreign Application Priority Data
Dec. 15, 2021 (TR) 2021/020041

(51) Int. Cl. A61K 31/704; A61K 31/196; A61K 31/4015; A61K 31/4174; A61K 31/4196; A61K 31/454; A61K 31/4725; A61K 31/506; G16B 15/30

(52) U.S. Cl. CPC A61K 31/704; A61K 31/196; A61K 31/4015; A61K 31/4174; A61K 31/4196; A61K 31/454; A61K 31/4725; A61K 31/506; G16B 15/30

(57) ABSTRACT

Disclosed are therapeutic molecules that can be used as an alternative to SHP2 phosphatase inhibitors, as a novel approach to targeting RAS isoforms with the G12D mutation that is involved in cancer formation.

### TECHNICAL FIELD

The present invention relates to therapeutic molecules that can imitate the dynamics of phosphorylated H-RAS protein, which can be used as an alternative to SHP2 phosphatase inhibitors that inhibit dephosphorylation of phosphorylated RAS protein, as a novel approach for targeting the H-RAS isoform harboring G12D mutation, which is involved in cancer formation. The invention also relates to a method for screening candidate molecules at the stage of therapeutic molecule development against cancer types in which the RAS isoform with the G12D mutation is predominant.

### STATE OF THE ART

RAS mutant proteins are responsible for nearly 30% of clinically observed cancer types. Therefore, the inhibitor development studies for RAS mutant proteins continue rapidly. The RAS protein family consists of 4 isoforms, namely H-RAS, K-RAS4A, K-RAS4B and N-RAS. Proteins in this protein family actively participate in signal transduction by interacting with cytoplasmic effector proteins. Therefore, they are responsible for the regulation of important cellular events such as cell proliferation, growth and differentiation.

In the light of this information, mutations in this protein family, especially the G12D mutation, are closely associated with cancer, since the inhibition of the regulation of these cellular events as a result of mutations transforms healthy cells into malignant forms. The close association of RAS mutant proteins with cancer has spurred studies focused on the development of inhibitors targeting these mutant proteins. However, unfortunately, no RAS mutant inhibitor could successfully complete clinical studies. The failure of inhibitor candidates proposed or developed so far has been associated with the inability to discover a ligand that can compete with the endogenous ligand of the protein and the absence of deep binding pockets on the surface of mutant RAS proteins.

Therefore, the emphasis has been focused on the discovery of allosteric sites, which can control protein function, as an alternative to the protein's nucleotide binding pocket, and some potential inhibitors have been brought into phase studies. More importantly, studies that have been conducted in recent years has shown that the function of RAS proteins can be modulated through post-translational modifications. Relatedly, phosphorylation of tyrosine residue number 32 of the H-RAS and N-RAS isoforms by Src kinase has been shown to inhibit the interaction of these isoforms with RAF. In addition, the K-RAS4B isoform has been shown to be phosphorylated by tyrosine residues number 32 and 64 by the same kinase and inhibit its interaction with RAF, as observed in H-RAS and N-RAS. Accordingly, it has been shown that the RAF/MEK/ERK signaling pathway, which is involved in cancer formation, is also terminated.

### OBJECT OF THE INVENTION

Until now, no drug development studies have been conducted in the literature that have been focused on imitating the modulations caused by Src kinase phosphorylation in the dynamics of the protein. Accordingly, one of the objects of the invention is to develop a method that can be used to detect H-RAS inhibitory agents that can imitate the changes in protein dynamics caused by Src kinase phosphorylation.

Another object of the invention is to develop molecules suitable for use as an H-RAS inhibitor agent.

### BRIEF DESCRIPTION OF THE INVENTION

The present invention includes H-RAS G12D, i.e., H-RAS with G12D mutation, cerubidine (Formula I), tranilast (Formula II), Nilotinib (Formula III), Imidafenacin (Formula IV), Epirubicin (Formula V), CHEMBL3490356 (Formula VI), ZINC16956714 (Formula VII), ZINC16382913 (Formula VIII), ZINC08648721 (Formula IX) molecules for use as inhibitor agent.

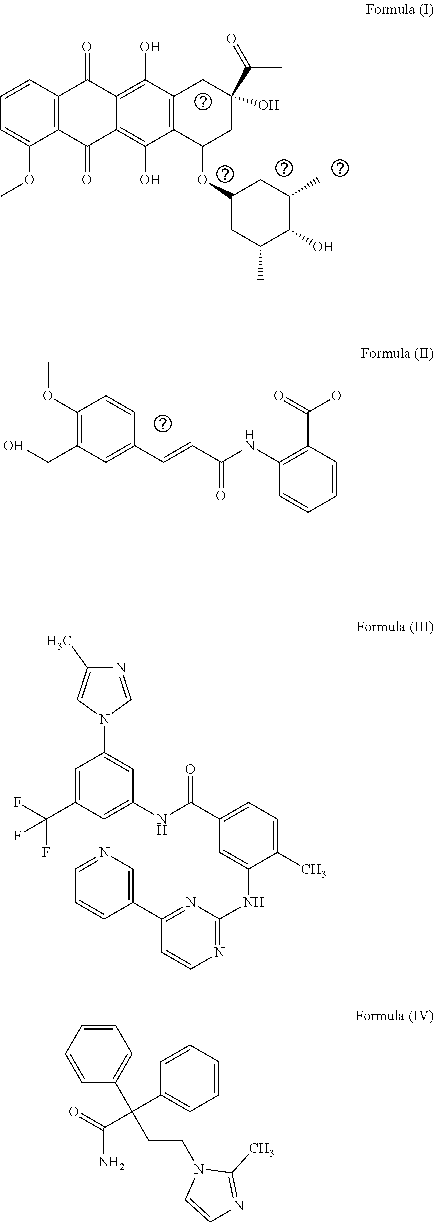

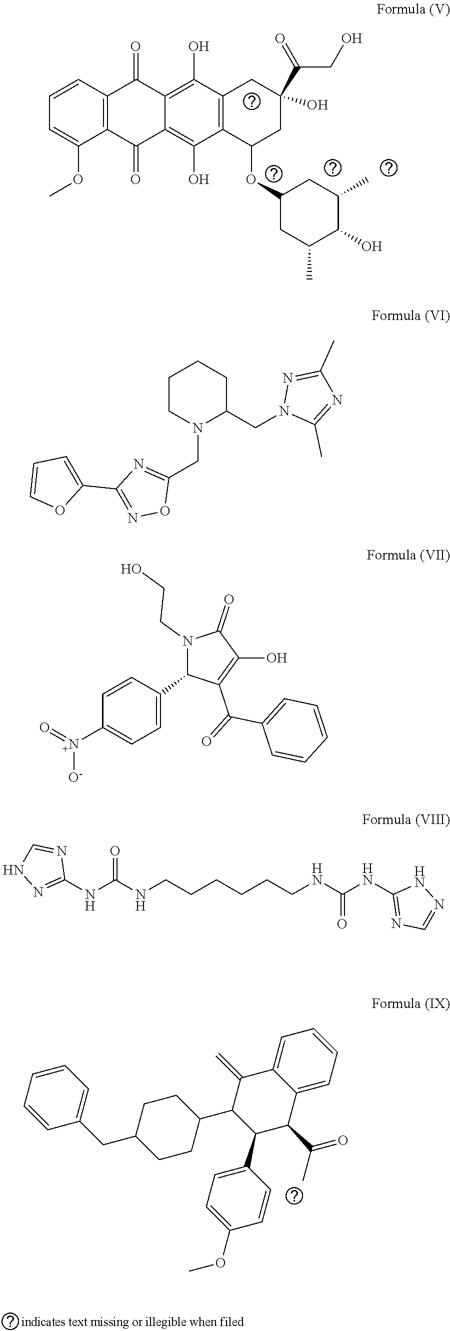

In another aspect, the invention relates to a method used to detect inhibitor molecules that can inhibit the interaction of mutant H-RAS with RAF by imitating phosphorylated H-RAS proteins, comprising the steps of;
i. Comparing the H-RAS G12D mutant, wild type H-RAS and phosphorylated H-RAS protein systems with molecular dynamics simulation,
ii. Clustering the trajectories of the above-mentioned systems after comparison,
iii. Modeling of pharmacophore groups for the binding pocket detected after clustering,
iv. Coupling of the candidate molecules to the binding pocket detected on the H-RAS G12D mutant protein,
v. Determining the stability of ligands with the appropriate binding pose and their effect on protein dynamics by molecular dynamics simulations,
vi. Detecting molecules that have the potential to disrupt the interaction surface of RAF and the mutant H-RAS G12D as inhibitor molecules.

In this context, molecules that can push Switch I away from the binding pocket by disrupting the interaction surface called Switch I will be considered successful.

### DESCRIPTION OF THE FIGURES

FIG. 1: Restriction enzyme digestion and PCR validation after the HRASwt & HRASG12D regions were cloned into lentiviral vectors by Gibson Assembly cloning method

FIG. 1a: GeneRuler 1 kb Plus DNA Ladder

FIG. 1b: Digestion control of Pico2-HRASwt plasmid DNA with BamHI and BlpI

FIG. 1c: PCR amplification of the HRASwt insert using plasmid DNA

FIG. 1d: Digestion control of Pico2 HRASG12D plasmid DNA with BamHI and BlpI

FIG. 1e: PCR amplification of HRASG12D insert using plasmid DNA

FIG. 2: RT-PCR validation of the established cell lines expressing wild type and G12D mutant HRAS proteins.

FIG. 2a: Total RNA isolated from cell lines after 24 hour plasmid transfection; then the RNA was converted to cDNA and RT-PCR was performed using the RAS primers expressions. GAPDH was used as a housekeeping gene.

FIG. 2b: Analysis result showing the intensity of the bands obtained as a result of RT-PCR compared to control cells.

FIG. 3: Showing the increase in protein levels with specific antibodies in cell lines with increased HRAS-G12D and HRAS-wt levels.

FIG. 4: Determination of cytotoxic doses of therapeutic molecules on cells by cell viability analysis

FIG. 5: Demonstration of successful precipitation of RAF-interacting RAS proteins (active RAS) with the Active RAS Pull Down Assay kit

FIG. 6: Cerubidine treatment with increasing doses in HRASG12D overexpressed HEK-293T cells

FIG. 7: a) Probability distribution of the distance measured between the side chain oxygen of tyrosine number 32 and the beta phosphate of GTP along the trajectories of the H-RAS systems b) Orientational dynamic of tyrosine number 32 of the phosphorylated H-RAS isoform along the trajectory.

FIG. 8: In the presence of a) Cerubidine, b) ZINC16382913, c) tranilast, d) nilotinib, e) ZINCZINC16956714, f) CHEMBL3490356, g) ZINC08648721, h) imidafenacinum, i) epirubicin and j) paroxetine, probability distribution of the distance measured between the side chain oxygen of tyrosine number 32 and beta phosphate of GTP of the H-RAS G12D mutant and the orientational dynamic to which tyrosine adapts.

### DETAILED DESCRIPTION OF THE INVENTION

The present invention includes H-RAS G12D, i.e., H-RAS with G12D mutation, cerubidine (Formula I), tranilast (Formula II), Nilotinib (Formula III), Imidafenacin (Formula IV), Epirubicin (Formula V), CHEMBL3490356 (Formula VI), ZINC16956714 (Formula VII), ZINC16382913 (Formula VIII), ZINC08648721 (Formula IX) molecules for use as inhibitor agent.

A preferred application of the invention relates to cerubidine (Formula I), tranilast (Formula II), Nilotinib (Formula III), Imidafenacin (Formula IV), Epirubicin (Formula V), CHEMBL3490356 (Formula VI), ZINC16956714 (Formula VII), ZINC16382913 (Formula VIII), ZINC08648721 (Formula IX) molecules for use as a therapeutic agent in the treatment of cancer disease in which the H-RAS G12D mutation is involved.

A particularly preferred application of the invention relates to cerubidine (Formula I), tranilast (Formula II), Nilotinib (Formula III), Imidafenacin (Formula IV), Epirubicin (Formula V), CHEMBL3490356 (Formula VI), ZINC16956714 (Formula VII), ZINC16382913 (Formula VIII), ZINC08648721 (Formula IX) molecules for use as a therapeutic agent in the treatment of thyroid neoplasm and/or ureteral carcinoma in which the H-RAS G12D mutation is involved.

An application of the invention relates to the cerubidine (Formula I) molecule for use as a therapeutic agent in the treatment of thyroid neoplasm and/or ureteral carcinoma in which the H-RAS G12D mutation is involved.

An application of the invention relates to the tranilast (Formula II) molecule for use as a therapeutic agent in the treatment of thyroid neoplasm and/or ureteral carcinoma in which the H-RAS G12D mutation is involved.

An application of the invention relates to the Nilotinib (Formula III) molecule for use as a therapeutic agent in the treatment of thyroid neoplasm and/or ureteral carcinoma in which the H-RAS G12D mutation is involved.

An application of the invention relates to the Imidafenacin (Formula IV) molecule for use as a therapeutic agent in the treatment of thyroid neoplasm and/or ureteral carcinoma in which the H-RAS G12D mutation is involved.

An application of the invention relates to the Epirubicin (Formula V) molecule for use as a therapeutic agent in the treatment of thyroid neoplasm and/or ureteral carcinoma in which the H-RAS G12D mutation is involved.

An application of the invention relates to the CHEMBL3490356 (Formula VI) molecule for use as a therapeutic agent in the treatment of thyroid neoplasm and/or ureteral carcinoma in which the H-RAS G12D mutation is involved.

An application of the invention relates to the ZINC16956714 (Formula VII) molecule for use as a therapeutic agent in the treatment of thyroid neoplasm and/or ureteral carcinoma in which the H-RAS G12D mutation is involved.

An application of the invention relates to the ZINC16382913 (Formula VIII) molecule for use as a therapeutic agent in the treatment of thyroid neoplasm and/or ureteral carcinoma in which the H-RAS G12D mutation is involved.

An application of the invention relates to the ZINC08648721 (Formula IX) molecule for use as a therapeutic agent in the treatment of thyroid neoplasm and/or ureteral carcinoma in which the H-RAS G12D mutation is involved.

As mentioned above, in another aspect, the invention relates to a method used to detect inhibitor molecules that can inhibit the interaction of mutant H-RAS with RAF by imitating phosphorylated H-RAS proteins, comprising the steps of;
i. Comparing the H-RAS G12D mutant, wild type H-RAS and phosphorylated H-RAS protein systems with molecular dynamics simulation,
ii. Clustering the trajectories of the above-mentioned systems after comparison,
iii. Modeling of pharmacophore groups for the binding pocket detected after clustering,
iv. Coupling of the candidate molecules to the binding pocket detected on the H-RAS G12D mutant protein,
v. Determining the stability of ligands with the appropriate binding pose and their effect on protein dynamics by molecular dynamics simulations,
vi. Detecting molecules that have the potential to disrupt the interaction surface of RAF and the mutant H-RAS G12D as inhibitor molecules.

In a preferred application of the method according to the invention, proteins and water molecules are modeled according to the CHARMM36 force field and TIP3P, respectively, to be used in molecular dynamics simulation in step (i).

The modeled systems were simulated in the NPT population in step (ii) of the method according to the invention under physiological conditions using the NAMD simulation package with at least 2 replicates. Trajectories from each replica were combined and (a) root-mean-square fluctuation values, (b) distance measurement between the side chain hydroxyl group of tyrosine number 32 and beta phosphate of GTP, and (c) principal components of GROMACS were analyzed using the “gmx rmsf”, “gmx distance”, “gmx covar” and “gmx anaig” modules. As a result of the comparison of the analysis outputs obtained for each system, the effect of phosphorylation on the structures and dynamics of isoforms was revealed. Subsequently, the trajectories of the G12D mutant systems were clustered according to the distance measured between (a) beta phosphate of guanosine triphosphate (GTP) and the side chain oxygen of threonine number 35, (b) beta phosphate of GTP and backbone amide number 60 of glycine and (c) the side chain oxygen of glutamine 61 and the beta phosphate of GTP. Thereby, the most frequently sampled conformation observed in the resulting trajectories of G12D mutant systems was determined.

Then, a possible binding pocket on the most frequently sampled conformation in step (iii) of the method according to the invention was determined for each G12D mutant system using Schrödinger's “SiteMap” module. Considering the chemical and orientational properties of the residues forming the determined binding pockets, pharmacophore groups were modeled using Schrödinger's “Develop pharmacophore Model” module.

Then, in step (iv) of the method, a library was created by obtaining molecules with a molecular weight less than 550 kDA and matching at least 3 pharmacophore groups modeled from ZINC15, DrugCentral, BindingDB, CHEMBL and NCGC databases. Each molecule in this created library was coupled to the binding pocket identified on the G12D mutant proteins using Schrödinger's “Glide SP” tool. The resulting coupling poses are evaluated according to the orientation of the ligand with respect to the GTP.

In step (v) of the method according to the invention, ligands that are oriented towards the nucleotide binding pocket from the ligands and do not overlap with the pocket, to which the GTP binds, are considered as potential candidate molecules. In this direction, the stability of protein-ligand complexes, which are thought to be successful, and the ability of ligands to modulate dynamics of mutant systems are elucidated by conducting molecular dynamics simulations under the above-mentioned conditions. After the molecular simulation processes, the inventors tested the identified candidate molecules by in vitro experiments.

HEK-293T cells, which are frequently used in gene transfer experiments, were preferred to investigate the in vitro effects of candidate molecules determined as a result of molecular dynamic analysis. In this context, to establish cell lines overexpressing the G12D mutant HRAS, firstly, the gene region encoding the relevant gene was cut from the commercially available bacterial expression plasmid and cloned into eukaryotic expression plasmids. The plasmids were then delivered into HEK-293T cells by transfection method. The cell lysates was then collected for RNA and protein isolation from these transgene delivered cells. RT-PCR and western blotting experiments confirmed that transgene delivery was achieved. By contrast, no G12D mutation was detected in wild type HEK-293T cells, therefore; they were considered and used as of negative control cells.

The candidate molecules determined as a result of molecular dynamics studies were applied to HEK-293T cells at different concentrations in order to determine the cytotoxic and optimum doses for the cells. Optimum dose ranges were determined for each molecule. HEK-293T/HRASG12D cells were treated with Cerubidine for 24 hours, one of these candidate molecules, and active RAS was examined due to the interaction of RAS protein in cells with RAF. In experiments carried out on this molecule; lysate was collected at different time points from cells treated with non-toxic concentrations of Cerubidine. Active RAS precipitation (Pull Down) and subsequent Western-blot experiments performed on these lysates were used to investigate the effect of Cerubidine molecule on active RAS at different time points for 12 hours in terms of G12D mutation specificity.

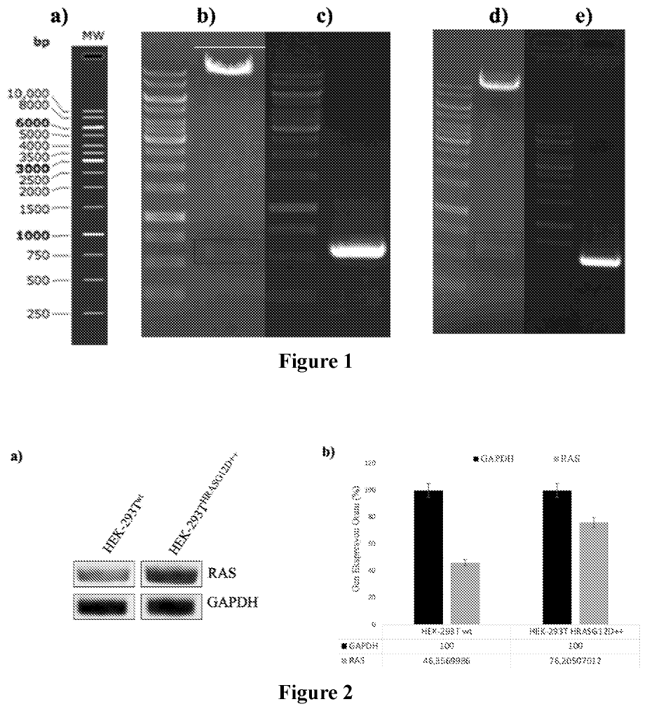

With this purpose, firstly, HRAS in the commercially available bacterial expression plasmid was transferred to the eukaryotic expression plasmid used in the laboratory by gibson cloning method to prepare wild and G12D mutatant HRAS expressing cell lines (FIG. 1).

The inventors also performed RT-PCR assay to establish cell lines expressing wild type and mutant RAS proteins. The results of this experiment were shown in FIG. 2.

Samples collected from transgene transduced and puromycin-resistant cells as well as HEK-293T wt cells without any gene transfer were used as controls in all experimental systems. The increase in the RNA level of HRAS carrying the G12D mutation in HEK-293T/HRASG12D-cells compared to cDNA obtained from control cells were shown in FIG. 2. In this experiment, GAPDH was used as a loading control, i.e. as a housekeeping gene, and increase in gene expression was normalized according to GAPDH.

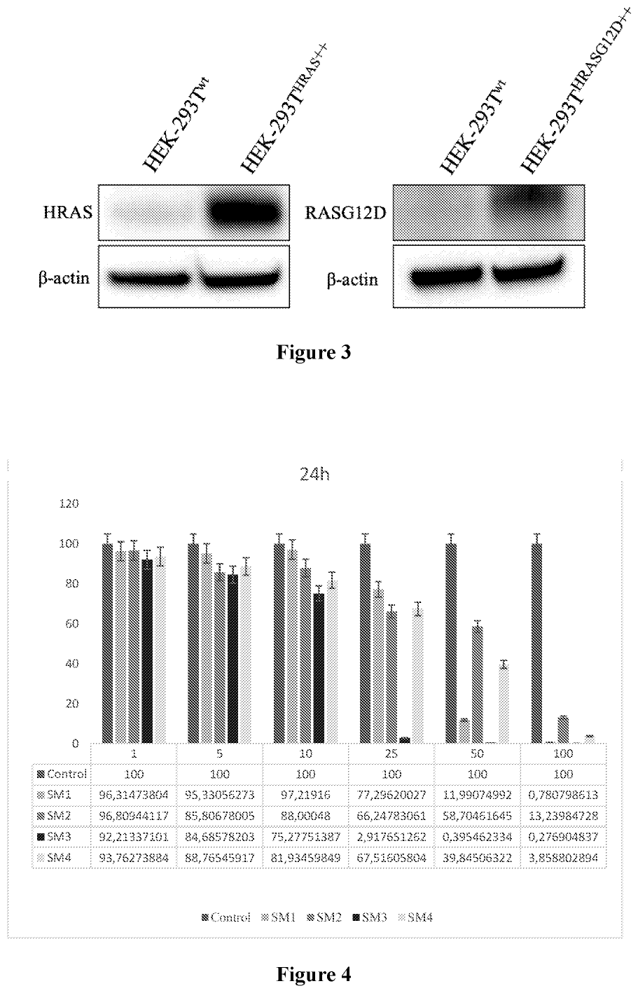

The inventors also conducted studies to show the increase in protein levels with specific antibodies in cell lines with increased HRAS-G12D and HRAS-wt levels. The results of this experiment are shown in FIG. 3.

In this study, protein isolation was performed from lysates obtained from cells within the scope of validating the increase in the expression levels of the relevant genes in transgene transferred HEK-293T cells by RT-PCR at the RNA level, and confirming the increase level at the protein level by western-blot. In this direction, the growth medium of the cells was discarded and washed using PBS. After washing, RIPA was added to each well and the cells were removed from the plate, then transferred to centrifuge tubes and incubated for 20 minutes at 25 rpm at +4 C. After the incubation, the supernatant was transferred to a new tube by centrifugation. The concentration of the obtained sample was measured by the NanoDrop device. According to measured values, the samples were treated with SDS and loaded onto the gel (Bolt™ 4 to 12%, Bis-Tris, 1.0 mm, Mini Protein Gel/Thermo). In western Blot experiments Invitrogen system (Mini Gel Tank & iBlot 2 Dry Blotting System) was used. After primary and secondary antibody treatment, the membrane was imaged with the ChemiDoc Imaging System (FIG. 3).

In the lysates collected from control cells, no band could be obtained for the RAS protein carrying the G12D mutation, but this increase was detected in the cells transfected with the vector carrying the HRAS gene containing the G12D mutation. After demonstrating the increase in HRAS-G12D protein levels, the increase in HRAS-wt (wild type HRAS) levels in lysates collected after transfection of wild-type HRAS-encoding lentiviral vectors into HEK-293T cells was demonstrated by HRAS-specific antibody.

Then, the inventors determined the cytotoxic doses of candidate molecules on cells by cell viability assays. Identified therapeutic molecules would be treated in further experiments with cells expressing the HRASG12D protein at high levels. Therefore, it is necessary to determine the cytotoxic effect of the molecules to be used on the cells and to determine the concentrations that will not create a toxic effect on the cells. Cell viability analysis experiment was carried out considering the the cytotoxic effect of therapeutic molecules determined previously by the inventors (mentioned above) on HEK-293T cells. One day before treatment with therapeutic molecules, HEK-293T cells were seeded on a 96-well costar black plate (CORNING) (high glucose DMEM containing 5% CO2 10% FBS at 370° C.). Therapeutic molecules (dissolved in DMSO) were added to the cells by increased concentrations from 0 to 100 uM. Cells were incubated for 24 hours under standard culture conditions. Cell viability was determined using the “CellTiterGlo Luminescent Cell Viability Assay (Promega)” and luminescent signals were measured by the SpectraMax i3 device. The luminescence signals from cells treated with therapeutic molecules were normalized against the signal from cells treated only with DMSO (FIG. 4).

SM1 in FIG. 4 represents the nilotinib molecule; SM2 represents the Paroxetine molecule; SM3 is the cerubidine molecule; and SM4 stands for epirubicin molecule. The said molecules are used as inhibitors of H-RAS G12D within the scope of the invention.

HEK-293T cells were treated with therapeutic molecules characterized as SM1, SM2, SM3 and SM4 (at concentrations of 1,5,10,25,50 and 100 uM). Cell viability analysis was performed after 24 hours of treatment with therapeutic molecules and the obtained values were plotted (FIG. 4).

The inventors also conducted a study to demonstrate that RAF interacting RAS proteins (active RAS) can be successfully precipitated with the Active RAS Pull Down Assay kit.

In the said study, HEK-293T cells with high expression of RAS proteins carrying the G12D mutation were used to examine the effect of candidate ligands on the interaction of H-RASG12D with RAF. First of all, an “Active Ras Pull-Down and Detection Kit (Thermo)” was provided to detect the interaction of the active RAS protein with RAF, and therefore the active RAS proteins involved in this interaction. Since the working principle of the kit is based on the precipitation of RAS proteins interacting with RAF, immunoprecipitation experiments were performed accordingly, since the use of the kit within the scope of this experiment was suitable for the target.

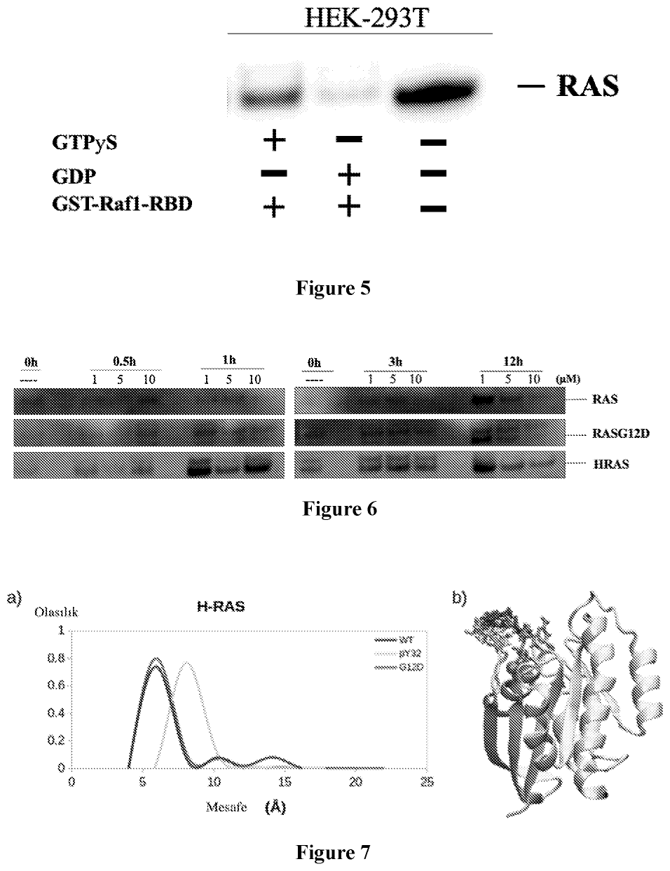

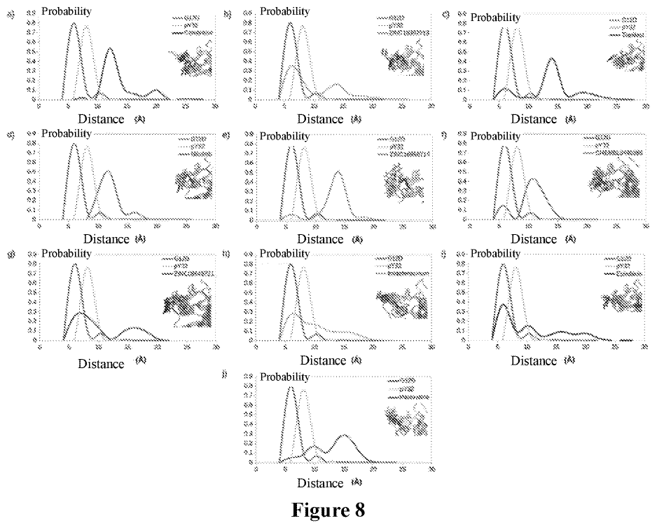

Firstly, protein isolation (500 ng-1000 ng) was performed from HEK-293T cells seeded in 6-well plates to demonstrate the proper functioning of the kit. The isolated proteins were first treated with GTPyS and GDP, and then bound to the glutathione-containing column with GST-Raf-RBD. Then, the presence of the RAS protein was examined by western blot experiment in the samples collected by elution from the column. The kit was found to work properly, with two control nucleotides, GTPyS and GDP, which can be used to generate positive and negative control lysates, respectively, showing the expected results (FIG. 5). Two control nucleotides of chitin of the lysates obtained in FIG. 5; binding to RAF1 protein after treatment with GTPyS (positive control) and GDP (negative control) was demonstrated by western blot.

Finally, the inventors treated HRASG12D overexpressed HEK293T cells with increasing doses of Cerubudine.

Then, established 293T-HRAS^{G12D} cells were treated with Cerubudine, one of the candidate molecules, at different doses (non-cytotoxic), and active RAS proteins were precipitated in the presence of RAF in the lysates collected from cells, following the kit protocol validated in FIG. 5. Then, active RAS, HRAS and RAS^{G12D} levels were analyzed by western blot at different time points. It was observed that 5 μM and 10 μM in cerubudine-treated cells caused a significant decrease in active RAS levels as an effective dose, and this reduction occurred in RAS carrying G12D mutant HRAS protein, not wild type HRAS levels. In addition, the most pronounced effect was detected in the group incubated with a dose of 10 μM at the end of 12 hours (FIG. 6).

Here, HRASG12D overexpressed cell lines (HEK-293T/HRAS^{G12D}) were treated with increasing doses of Cerubudine and lysate was obtained for protein isolation at different time points (0.5 h, 1 h, 3 h, 12 h). Active RAS precipitation was performed using isolated proteins and RAS, RASG12D and HRAS protein levels were examined by Western blot. Within the scope of the results obtained, there was a decrease in active RAS protein levels depending on the dose increase, especially in the 12th hour treated samples. Cerubidine treatment in cell lines overexpressing HRASG12D reduced the interaction of active RAS protein with RAF. The decrease in active RAS levels in the membrane with G12D specific antibody supports that this effect is mutant specific.

### EXAMPLES

Example 1: Method for detecting inhibitor molecules that can inhibit the interaction of mutant H-RAS with RAF by imitating phosphorylated H-RAS proteins according to the invention.

As mentioned in step I of a method used to detect inhibitor molecules described within the scope of the invention, the effects of G12D mutation and Src kinase-mediated tyrosine phosphorylation on the structure and dynamics of the H-RAS isoform were investigated through molecular dynamic simulations.

In a preferred application of the method according to the invention, proteins and water molecules were modeled according to the CHARMM36 force field and TIP3P, respectively, to be used in molecular dynamic simulation. The modeled systems were simulated in the NPT ensemblen under physiological conditions with at least 2 replicates using the NAMD simulation package. Trajectories from each replica were combined and (a) root-mean-square fluctuation values, (b) distance measurement between the side chain hydroxyl group of tyrosine number 32 and beta phosphate of GTP, and (c) principal components of GROMACS were analyzed using the “gmx rmsf”, “gmx distance”, “gmx covar” and “gmx anaig” modules. As a result of the comparison of the analysis outputs obtained for each system, the effect of phosphorylation on the structures and dynamics of isoforms was revealed. Subsequently, the trajectories of the G12D mutant systems were clustered according to (a) beta phosphate of guanosine triphosphate (GTP) with the side chain oxygen of threonine number 35, (b) beta phosphate of GTP with backbone amide number 60 of glycine and (c) measured distances between the side chain oxygen of glutamine number 61 and the beta phosphate of GTP. Thereby, the most frequently sampled conformation along the obtained trajectories of the G12D mutant systems was determined. Then, a possible binding ability on the most frequently sampled conformation was determined for each G12D mutant system using Schrödinger's “SiteMap” module. Considering the chemical and orientational properties of the residues forming the determined binding pockets, pharmacophore groups are modeled using Schrödinger's “Develop pharmacophore Model” module. Then, a library was created by obtaining molecules with a molecular weight less than 550 kDA and matching at least 3 pharmacophore groups modeled from ZINC15, DrugCentral, BindingDB, CHEMBL and NCGC databases. Each molecule in this created library is coupled to the binding pocket identified on the G12D mutant proteins using Schrödinger's “Glide SP” tool. The resulting coupling poses were evaluated according to the orientation of the ligand with respect to the GTP. Ligands that are oriented towards the nucleotide binding pocket from the ligands and fail to show overlap with the pocket to which GTP binds were evaluated as promising data. In this direction, the stability of protein-ligand complexes, which are thought to be successful, and the ability of ligands to simulate mutant systems to systems in the phosphorylated state are elucidated by conducting molecular dynamics simulations under the above-mentioned conditions.

### INDUSTRIAL APPLICABILITY

The molecules described within the scope of the invention are those that have previously been clinically studied for different indications and were found to be non-toxic. Thanks to the invention, the use of these molecules as therapeutic agents against different indications is provided.

In addition, thanks to the method described within the scope of the invention, it will be possible to obtain many other active substances in the future in this field, where there are little known therapeutic agents. In this respect, the invention concerns both end users in the health sector and medication developers.

1. H-RAS (H-RAS G12D) with G12D mutation, cerubidine (Formula I) or tranilast (Formula II) or Nilotinib (Formula III) or Imidafenacin (Formula IV) or Epirubicin (Formula V) or CHEMBL3490356 (Formula VI), ZINC16956714 (Formula VII) or ZINC16382913 (Formula VIII) or ZINC08648721 (Formula IX) molecules for use as inhibitor agent, wherein they are shown as follows;

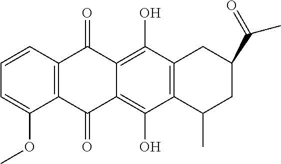 [SMILES: COc1cccc2c1C(=O)c1c(O)c3c(c(O)c1C2=O)C[C@@H](C(C)=O)CC3C]

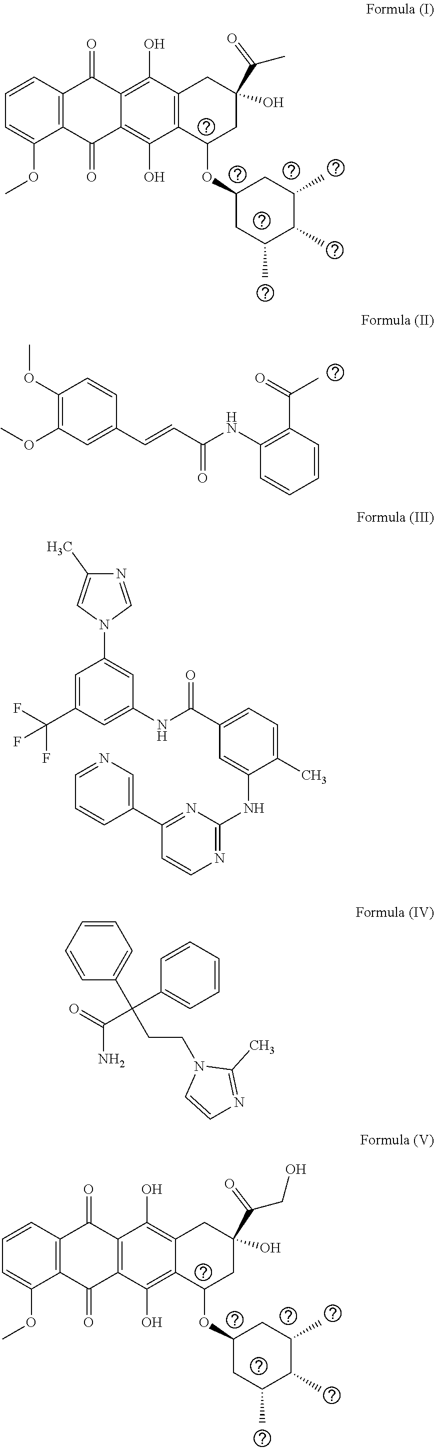

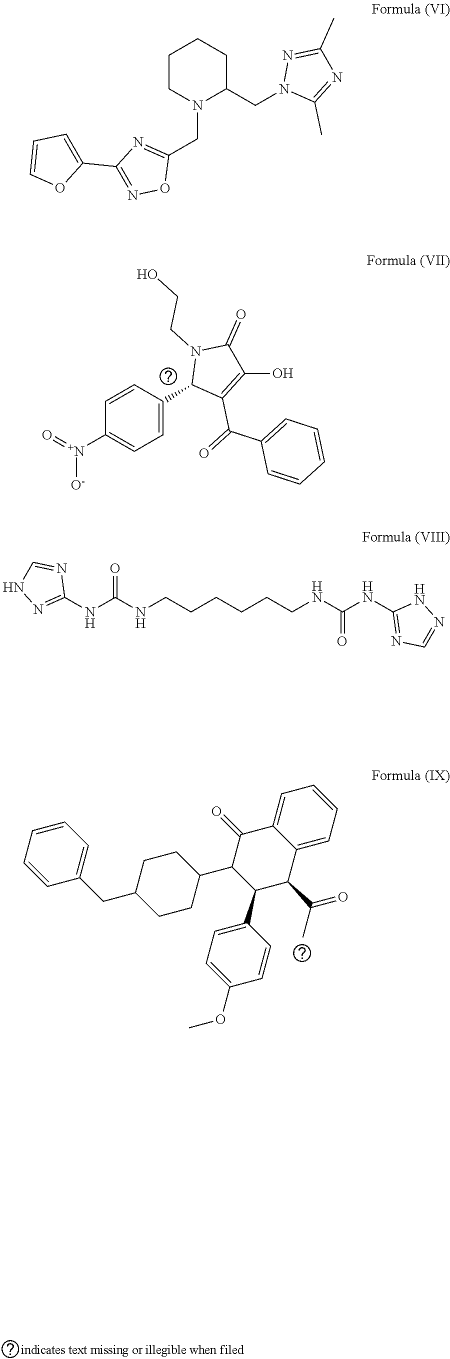 [SMILES: Cc1nc(C)n(CC2CCCCN2Cc2nc(-c3ccco3)no2)n1 ; O=C(C1=C(O)C(=O)N(CCO)[C@H]1c1ccc([N+](=O)[O-])cc1)c1ccccc1 ; O=C(NCCCCCCNC(=O)Nc1ncn[nH]1)Nc1nc[nH]n1 ; COc1ccc([C@H]2C(C3CCC(Cc4ccccc4)CC3)C(=O)c3ccccc3[C@H]2C(C)=O)cc1]

2. The use according to claim 1, wherein cerubidine (Formula I), tranilast (Formula II), Nilotinib (Formula III), Imidafenacin (Formula IV), Epirubicin (Formula V), CHEMBL3490356 (Formula VI), ZINC16956714 (Formula VII), ZINC16382913 (Formula VIII), ZINC08648721 (Formula IX) molecules for use as a therapeutic agent in the treatment of cancer disease in which the H-RAS G12D mutation is involved.

3. The use according to claim 2, wherein the cancer disease in which the H-RAS G12D mutation is involved is thyroid neoplasm and/or urothelial carcinoma.

4. A method used for detecting inhibitor molecules that can inhibit the interaction of mutant H-RAS with RAF by imitating phosphorylated H-RAS proteins, comprising the steps of;
i. Comparing the H-RAS G12D mutant, wild type H-RAS and phosphorylated H-RAS protein systems with molecular dynamics simulation, Clustering the trajectories of the H-RAS G12D mutant, wild type H-RAS and phosphorylated H-RAS protein systems after comparison,
ii. Modeling of pharmacophore groups for the binding pocket detected after clustering,
iii. Coupling of the candidate molecules to the binding pocket detected on the H-RAS G12D mutant protein,
iv. Determining the stability of ligands with the appropriate binding pose and their effect on protein dynamics by molecular dynamics simulations,
v. Detecting molecules that have the potential to disrupt the interaction surface of RAF and the mutant H-RAS G12D as inhibitor molecules.

5. A method according to claim 4, characterized in that the proteins and water molecules are modeled according to the CHARMM36 force field and TIP3P, respectively, for molecular dynamics simulation in step (i).

6. A method according to claim 4, characterized in that the trajectories of the G12D mutant systems are clustered according to the distance measured between (a) beta phosphate of guanosine triphosphate (GTP) and the side chain oxygen of threonine number 35, (b) beta phosphate of GTP and backbone amide number 60 of glycine and (c) the side chain oxygen of glutamine number 61 and the beta phosphate of GTP in step (ii).

7. A method according to claim 4, characterized in that a possible binding pocket on the most frequently sampled conformation is determined for each G12D mutant system using Schrödinger's “SiteMap” module in step (iii), and considering the chemical and orientational properties of the residues forming the determined binding pockets, pharmacophore groups are modelled by using Schrödinger's “Develop pharmacophore Model.”

8. A method according to claim 4, characterized in that a library of molecules with a molecular weight of less than 550 kDA and matching at least 3 pharmacophore groups modeled from molecular databases such as ZINC15, DrugCentral, BindingDB, CHEMBL and NCGC is created in step (iv), and then each molecule in the library is coupled to the binding pocket determined on the G12D mutant proteins using Schrödinger's “Glide SP” tool.

9. A method according to claim 8, wherein the coupling pose is determined according to the orientation of the ligand with respect to GTP.

10. A method according to claim 4, characterized in that ligands that are oriented towards the nucleotide binding pocket from the ligands and do not overlap with the pocket to which the GTP binds in step (v) are considered as potential candidate molecules.
